(19) United States
(12) Patent Application Publication
(10) Pub. No.: US 2022/0213433 A1
(43) Pub. Date: Jul. 7, 2022

(54) Three-Dimensional Cross-Linked Scaffolds of Cord Blood Plasma and Their Use

(71) Applicant: SANFORD HEALTH, Sioux Falls, SD (US)

(72) Inventors: Michelle BAACK, Sioux Falls, SD (US); Pilar DE LA PUENTE, Sioux Falls, SD (US); Tyler GANDY, Sioux Falls, SD (US)

(21) Appl. No.: 17/612,147

(22) PCT Filed: Jun. 15, 2020
(86) PCT No.: PCT/US2020/037709

§ 371 Date: Nov. 17, 2021

Related U.S. Application Data
(60) Provisional application No. 62/860,967, filed on Jun. 13, 2019.

(51) Int. Cl. C12N 5/00; C12N 5/073

(52) U.S. Cl. CPC C12N 5/0068; C12N 5/0062; C12N 5/0605; C12N 2513/00; C12N 2537/10; C12N 2500/33; C12N 2500/14; C12N 2535/00; C12N 2533/90

(57) ABSTRACT

The disclosure provides three-dimensional cross-linked scaffolds generated from cord blood plasma, and methods for making and using such scaffolds.

### CROSS REFERENCE

This application claims priority to U.S. Provisional Patent Application Ser. No. 62/860967 filed Jun. 13, 2019, incorporated by reference herein in its entirety.

### FEDERAL FUNDING STATEMENT

This invention was made with government support under Grant Nos. NIH/NIGMS 5 P20 GM103548-08 and NIH/NIGMS 2 P20 GM103620-06 awarded by the National Institutes of Health. The government has certain rights in the invention.

### BACKGROUND

Understanding the role of maternal health or the safety of drugs during pregnancy on early human development is an unmet need due to the high-risk status of this patient population. Animal models to understand embryonic and fetal development or test drug safety are expensive and they often have limited translation to human disease. Currently, there is no ethically acceptable human model that adequately mimics the in vivo developmental environment in a precision based way. Specifically, the effects of new or even commonly used, but untested medications, pollutants, or other molecular compounds are of particular relevance to women of reproductive age; especially when their effects on fetal health are unknown.

### SUMMARY OF THE DISCLOSURE

In one aspect, the disclosure provides methods comprising:

(a) mixing cord blood plasma, with cross-linker and stabilizer to form a mixture; and

(b) incubating the mixture for a time and under conditions to form a three-dimensional cross-linked scaffold. In one embodiment, the method comprises pre-mixing the cord blood plasma with biological cells to form a pre-mixture, wherein the pre-mixture is mixed with the cross-linker and stabilizer. In one embodiment, the cord blood plasma comprises cord blood plasma obtained from a subject having maternal pregnancy complications such as, but not limited to Type 1, Type 2 or gestational diabetes, preeclampsia, maternal obesity, smoking, multiple gestation, or preterm labor, and/or a subject having fetal pregnancy complications such as birth defects, chromosomal or hereditary disorders or intrauterine growth disturbance. In another embodiment, the biological cells comprise normal or aberrant stem cells from any suitable source, including but not limited to inducible pluripotent stem cells (iPSC), embryonic stem cells, fetal stern cells, hematopoietic stem cells, mesenchymal stem cells, bone marrow derived stem cells, umbilical cord derived stem cells, or placenta derived stem cells.

In one embodiment, the cross-linker comprises a cross-linker selected from the group consisting of calcium chloride and thrombin, or a combination thereof. In another embodiment, the stabilizer comprises tranexamic acid. In a further embodiment, no exogenous polymer is present in the three-dimensional cross-linked scaffold.

In another aspect, the disclosure provides three-dimensional cross-linked scaffolds comprising cord blood plasma. In one embodiment, the scaffold further comprises biological cells within the scaffold. In a further embodiment, the cord blood plasma comprises cord blood plasma obtained from a subject having maternal pregnancy complications such as Type 1, Type 2 or gestational diabetes, preeclampsia, maternal obesity, smoking, multiple gestation, or preterm labor, and/or a subject having fetal pregnancy complications such as birth defects, chromosomal or hereditary disorders or intrauterine growth disturbance. In one embodiment, the biological cells comprise normal or aberrant stem cells from any suitable source, including but not limited to inducible pluripotent stem cells (iPSC), embryonic stem cells, fetal stem cells, hematopoietic stein cells, mesenchymal stem cells, bone marrow derived stem cells, umbilical cord derived stem cells, or placenta derived stem cells. In further embodiments, the scaffolds comprise a cross-linker selected from the group consisting of calcium chloride and thrombin, or a combination thereof, and/or comprise tranexamic acid as a stabilizer. In another embodiment, no exogenous polymer is present in the three-dimensional cross-linked scaffold.

In a further aspect, the disclosure provides methods for using the three-dimensional cross-linked scaffolds for any suitable purpose, including but not limited to drug screening, tissue engineering, cell differentiation, toxicology studies including reproductive toxicology/teratogenicity studies, cell fate studies based on exposure to stimuli, inherent cell abnormalities, developmental biology, developmental origins of disease, regenerative medicine, etc. In one embodiment, the methods comprise

(a) contacting the three-dimensional cross-linked scaffold with a test moiety, wherein the test moiety may include, but is not limited to a drug, toxin, hormone, cytokine, small molecule, and/or other stimulus;

(b) culturing the cells of interest within the scaffold; and

(c) determining an effect of the test moiety on the cells of interest.

### DESCRIPTION OF THE FIGURES

FIG. 1. Fibrinogen content in cord blood plasma is lower than peripheral blood plasma and higher in diabetic subjects, highlighting precision-based applications. Fibrinogen levels (mg/dL) present in cord blood plasma from non-diabetic and diabetic subjects. *p<0.05 by t-test.

FIG. 2(a-b). Chemical characterization of cord blood plasma 3D culture model referred as InvitroWOMB (iWOMB). (a) A measurement of the time (minutes) to achieve matrix cross-linking using two relevant cross-linking agents of the blood coagulation process including CaCl₂ (0-10 mg/ml) and Thrombin (0-5 mg/ml); (b) Stabilization effect studies of preventing fibrin degradation and stability improvement in the scaffold were achieved by testing the antifibrinolytic agent tranexamic acid (0-5 mg/ml). **p<0.001 compared to lack of stabilizer by t-test.

FIG. 3(a-b). Physical characterization of iWOMB. (a) iWOMB blank (acellular) scaffold stiffness revealed a soft gel. Scaffolds become stiffer with cells due to cellular contribution of collagen to the ECM. (b) Representative fluorescent images exhibit increased expression of collagen I and collagen III at day 4 for cell-seeded iWOMB cultures compared to blank gels; Scale bar=100 μm.

FIG. 4(a-e). Cell properties within iWOMB. (a) Despite the gelatinous nature, (b) three-dimensional umbilical cord plasma-derived scaffolds remain porous as shown by scanning electron microscopy (SEM) of this acellular scaffold. Cells within scaffolds can be imaged (c) fixed or (d) live, which demonstrates cells remain viable and retain organelle structure in the scaffold. (c) Umbilical cord blood plasma was combined with human umbilical cord derived mesenchymal stem cells (hu-MSCs), MSC media, crosslinking, and stabilizer solutions. In a 24-well plate, 1 ml of the combined solution was aliquoted into each well. The iWOMB solution was allowed to crosslink for approximately 10 minutes before 1 ml of stem cell media was slowly added to the tops of the iWOMBs. 20K hu-MSC were incorporated into each well and cultured at 37° in 5% CO₂. (c) Samples were collected, stained and prepared for serial block-face microscopy by immersion in 2% glutaraldehyde+2% paraformaldehyde in 0.15 M cacodylate buffer containing 2 mM calcium chloride until further processed (minimum of 24 hr.). Fixed samples were processed and embedded in polyepoxide resin Durcapan™ (EMS, Hatfield, Pa.). High resolution block-face images were obtained using VolumeScope™ serial block-face SEM (Thermo Fisher, Waltham, Mass.). A stack of approximately 500 block-face images (50 nm) were obtained then aligned and filtered using Amira software (Thermo Fisher, Waltham, Mass.). (d) After incubation, hu-MSC in iWOMB were stained with 1.43 uM MitoTracker™ green (M7514, Thermo Fisher Scientific), 2 uM LysoTracker™ red (ThermoFisher, Waltham, Mass.), and 1:200 Hoescht (AS-83218, AnaSpec Inc.). Confocal live-cell images for morphology were acquired at 60× using a Nikon AIR Confocal microscope. Hoescht stained nuclei and long tubular, dynamic mitochondria indicate good viability in conditions. Human and non-human stem cells can be used in the cord plasma derived scaffold at varying density. (e) 3T3 mouse embryonic fibroblast stem cells were imaged in 96 well plates at seeding densities of 2-200K using an EVOS™ Cell Imaging System at 10× cells immediately after plating (top) and after 24 hours in culture (bottom) to demonstrate incorporation in to cord plasma-derived scaffolds. Scale bar, 400 um. Hu-MSC and T3Ts were cultured in αMEM, 10% FBS, 1%1-glutamine, 1% pen/strep at 37° with media changes every 2-3 days depending on the application. High-quality images by SEM (4b & c) or other fixed IHC prep can also be obtained by plating cellular and acellular iWOMB in Beem® capsules (FIG. 10) to allow fixing, sectioning and staining without losing orientation.

FIG. 5(a-c). Cell-to-cell and cell-to-matrix organization within three-dimensional cord plasma derived scaffolds during cardiogenesis. Confocal live cell image of hu-MSC treated with 5-azacytadine (5′AZA) for cardiogenesis were plated in iWOMB (20K cells/1000 μl mixture) in StemPro™ Cardiogenic Differentiation media B and M and imaged over time. (a) After 24 hours post-5′AZA treatment at 37° C., cells within iWOMB were stained with MitoTracker™ green, TMRE, and Hoescht. A representative 60× image of two cardiac progenitors shows cell to cell interactions shortly after plating. (b-c) Representative three-dimensional Z-stack images of hu-MSC derived cardiac progenitors on day 13 of differentiation were reconstructed using Nikon NIS analysis software. (b) Top and (c) side views show at least 7 densely packed cell layers despite 5′ AZA treatment and long-term culture in various media (StemPro™ Cardiogenic Differentiation media B and M).

FIG. 6(a-d). Stem cell growth and multi-lineage differentiation within iWOMB. Hu-MSC can undergo multi-lineage differentiation in iWOMB. (a) derived cardiac progenitors in iWOMB are bi-nucleated, more rod-shaped, and stain positive for cardiomyocyte specific myosin light chain 2 (MLC2v, green) and cardiac troponin (TNNT2, red) at 13 days post 5′AZA. (b) Hu-MSC derived adipocyte is stained with Oil-red-O to demonstrate significant lipid droplet accumulation 14 days after differentiation with StemPro™ Adipogenesis Differentiation kit (Gibco, A10070-01). (c) Hu-MSC derived osteocytes are densely packed and stain intensely positive with alizarin red on day 7 after differentiation with StemProm™ Osteogenesis Differentiation Kit (Gibco, A10072-01). (d) Confocal imaging of live cells in iWOMB demonstrates ultrastructural changes during biological development. Here MitoTracker™ (mitochondria), TMRE (MMP well charged mitochondria) and Hoescht (nuclei) stained hu-MSC and derived cardiac progenitors from day 0 to 13 of differentiation depict developmental sub-cellular organization of subsets of perinuclear and interfibrillar mitochondria that are unique to myocytes. Confocal images at 60×.

FIG. 7(a-c). Protein and RNA isolation from iWOMB. Hu-MSC from one subject (98) were cultured on unmatched cord blood derived scaffolds (iWOMB) or collagen coated 24 well plates and differentiated in to multiple lineages. On advancing days of differentiation (D), cells were collected and protein was isolated and quantified by DC Protein Assay (BioRad, Hercules, Calif.). (a) Bar graphs represent total protein collected from cell lysate within iWOMB (left) and collagen coated plates (right). Cells were from the same patient and differentiated using the same methods/kit. Data demonstrates that protein from cell-seeded iWOMB is typically greater or equal to protein recovered from cells on collagen coated plates and reflects expected cell numbers during cardiogenic, osteogenic and adipogenic differentiation. (b) Hu-MSC were seeded to cord plasma derived iWOMBs in 96-well plates at increasing seeding density from 20K to 100K cells/scaffold. RNA was isolated from cell pellets and measured by Epoch spectrophotometer (BioTek, Winooski, Vt.). Bar graphs depict total RNA recovered from cell-seeded iWOMB and demonstrates increasing RNA yield up to 80K cells; thereafter yield decreases, likely due to cell die off from overcrowding that was observed on the plate. (c) RNA obtained from cell-seeded iWOMB was converted to cDNA for qPCR; as expected, there is a relative rise in myocyte enhancer factor 2C (MEF2C) from D2 to 14 during hu-MSC cardiogenic differentiation.

FIG. 8(a-c). Developmental microenvironment (DME) of iWOMB. Protein (100 ug) isolated from pelleted cells and/or the supernatant was incubated on a custom membrane human antibody array (Ray Biotech, Peachtree Corners, Ga.) to measure cytokines and growth factors within the DME of acellular and cell-seeded iWOMB. (a) The membrane detects relative expression compared to negative and positive controls as detailed and demonstrated. Following overnight incubation, membranes were exposed and imaged. Individual expression relative to four negative controls (membrane background) was calculated by densitometry. Differences in proteins (in duplicate) from pelleted hu-MSC undergoing cardiac differentiation and collected supernatant were compared using 1-way ANOVA with Dunnett post-test to compare expression at day 0 (hu-MSC plated), 2, 5, 7, and 10 to baseline (acellular iWOMB+media). (b) While there was little difference in protein from cell pellets, the supernatant (pictured here) demonstrated dynamic changes in the DME. Bar graphs represent relative expression of individual proteins at each time point. Cytokines within the DME were both cell and time dependent. IL-6 was only present in cell-seeded iWOMB and remained consistently higher. TNFα and IL-10 increased with days in culture. Growth factors within the DME were media (media changes initially captured on day 2 and 5) and cell dependent (increase over time). (c) These differences are highlighted further by bar graphs that represent TNFα expression overtime in both cell and supernatant protein fractions. *p<0.05 compared to baseline (acellular iWOMB+media) by 1-way ANOVA and Dunnett post-test: p<0.05. ∧p<0.05 cell lysate is different than supernatant by T-test.

FIG. 9(a-d). Precision capabilities. (a) Using a custom human antibody array we measured relative expression of cytokines and growth factors in cord plasma for scaffolds from subjects with Type 1 (T1D), Type 2 (T2D) and gestational diabetes (GDM). Bar graphs represent relative expression by densitometry compared to negative control (membrane background). (b) Hu-MSC from control, T1D, T2D and GDM donors (n=2-3/group) were uniformly plated (50K live cells/well) in stem cell media to collagen-coated, 24-well plates. Trypan blue was used to count live cells every 24 hours and growth was measured and compared. Bar graphs represent fold change from baseline to 72 hours; *p<0.05 by one-way ANOVA with Dunnett post-test, (c) Control hu-MSCs were treated with metformin at increasing doses (0, 25, 50 and 100 μM) and growth was followed as previously described. Growth curves show the number live cells counted at each time point and illustrates that metformin impairs hu-MSC growth in a dose dependent manner (d) The effect of a translatable dose of metformin (25 μM) on stem cell growth was evaluated in control and GDM-exposed hu-MSC (n=2-3/group). Bar graphs represent the number of cells/well remaining at 72 hours after initial plating of 20K cells/well. GDM-exposed cells had a trend towards slower growth, but metformin increased growth so that 72 hour cell counts were close to that of controls.

FIG. 10(a-d). Function and scalability. iWOMB is suitable for a wide range of applications depending on the needs of the study. Acellular and cellular assays have been done in 24-well, 96-well, 4-well glass chamber slides and Beem® embedding capsules. (a) Photograph of hu-MSC derived osteocytes in three-dimensional cord plasma scaffolds within 24-well plates demonstrates optimal size for differentiation as osteogenesis can be seen by day 7 when a visible white layer of calcium forms in the wells. (Pink color in the 2 right wells indicates fresh media). (b) Photograph of cell-seeded iWOMB in Beem® capsules shows that these applications are ideal for fixed, embedded and sectioned images or tissue regeneration studies where specified orientation is necessary. (c) Top and side view of 4-well chamber slides with pre-cross-linked cord plasma derived scaffolds demonstrates the three dimensional nature and how confocal live cell imaging or fixed organizational imaging can be best accomplished in these. (d) As shown, the smaller size of 96-well plates requires less plasma, media and cells to create iWOMB. This allows upscaling that may be useful for high-throughput drug screening, however less RNA and protein can be isolated especially during differentiation when proliferation declines. Bar graphs show total RNA isolated from iWOMB in 24-well and 96-well plates at day 2, 7, 14 and 21 following cardiogenic differentiation as detailed above. To optimize RNA recovery during differentiation, hu-MSC were seeded at increasing density (20k/well to 100k/well) in 96-well plates. Bar graphs show RNA recovery at day 2, 14 and 21 for each original seeding density.

### DETAILED DESCRIPTION

As used herein, the singular forms “a”, “an” and “the” include plural referents unless the context clearly dictates otherwise. All embodiments of any aspect of the disclosure can be used in combination, unless the context clearly dictates otherwise.

As used herein, “about” means +/−5% of the recited parameter.

In a first aspect, the disclosure provides methods, comprising:

(a) mixing cord blood plasma with cross-linker and stabilizer to form a mixture; and

(b) incubating the mixture for a time and under conditions to form a three-dimensional cross-linked scaffold.
This disclosure provides a tissue-like 3D scaffold that utilizes cord plasma as the matrix supporting the recapitulation of maternal-fetal exposures and cellular interactions and the tissue architecture without the use of exogenous materials. The cord blood plasma contains fibrinogen, a plasma glycoprotein involved in the blood coagulation process. The cord blood plasma contains a personalized set of pro-inflammatory cytokines, and growth factors that vary based on maternal, placental and fetal health and interactions. The scaffolds disclosed herein are reproducible models that tests cell fate in varied developmental microenvironments. This cost effective, ethically acceptable, high-throughput platform can be used to test the effects of various exposures on human development while also accounting for maternal or fetal based health conditions. Overcoming this hurdle is a rate limiting step for developing new or repurposed medications for use in pregnant women and newborns. For example, using stem cells from any appropriate source, including human umbilical cord derived mesenchymal stem cells cultured on the scaffolds allows developmental and reproductive toxicology testing in a tissue-specific, dose- and time-controlled environment. The 3D scaffold generated for use in the platform provides several unique advantages to other natural or synthetic matrices. It is derived from cross-linked umbilical cord blood, providing a source of nutrients, growth factors and cytokines that mimic the developmental microenvironment present in utero. The scaffold enables three-dimensional culture of, for example, mesenchymal stem cells, with cell-to-cell and cell-to-matrix interactions present. It can be used to test medications for a pre-identified population of patients by pairing normal or diseased stem cells (with their inherent genetic and epigenetic predisposition) and cord blood (with altered cytokines and growth factors) from a group of patients from the desired population.

The cord blood plasma may be freshly prepared, may be thawed from frozen samples, or may be obtained via any other suitable technique. The cord blood plasma may be obtained from any suitable source including collection during or following an uncomplicated or complicated pregnancy. In various embodiments, the subject may have maternal pregnancy complications such as Type 1, Type 2 or gestational diabetes, preeclampsia, maternal obesity, smoking, multiple gestation, or preterm labor. The subject may also have fetal pregnancy complications such as birth defects, chromosomal or hereditary disorders or intrauterine growth disturbance. The three-dimensional cross-linked scaffolds can be used, for example, for drug screening, tissue engineering, cell differentiation, toxicology studies including reproductive toxicology/teratogenicity studies, cell fate studies based on exposure to stimuli, inherent cell abnormalities, developmental biology, developmental origins of disease, regenerative medicine, etc.

In one embodiment, the method comprises pre-mixing the cord blood plasma with biological cells to form a pre-mixture, wherein the pre-mixture is mixed with the cross-linker and stabilizer. The pre-mixing of cord blood plasma with biological cells to form a pre-mixture may be carried out under any suitable conditions. In one embodiment, the pre-mixing is carried out at room temperature.

Any suitable biological cells may be used as deemed appropriate for an intended use. Cord blood plasma and the resulting three-dimensional cross-linked scaffolds can be used with normal or aberrant stem cells from any suitable source, including but not limited to inducible pluripotent stem cells (iPSC), embryonic, fetal, hematopoietic, mesenchymal, bone marrow derived, umbilical cord derived, or placenta derived stem cells in order to test mechanisms of normal or abnormal biologic development or screen therapeutic compounds for efficacy or developmental toxicity. In some embodiments, the cells and cord plasma are matched (i.e.: from the same subject). They may also be unmatched plasma and biological cells, or matched or unmatched combinations of plasma and biological cells from more than one subject may be used. The mixing of cord blood plasma with biological cells to form a mixture may be carried out under any suitable conditions. In one embodiment, cord plasma and resulting scaffolds from normal (non-complicated pregnancy) and abnormal pregnancy may be used to study cellular responses following exposure to normal or abnormal circulating factors including, but not limited to nutrients, fuels, hormones, cytokines, adipokines, eicosanoids, or hormones. In another embodiment, normal or abnormal cord blood plasma with circulating or added drug compounds or small molecules can be used to test responses of normal or abnormal stem cells to potential therapeutics or toxicants under variable developmental conditions.

The biological cells may be present at any suitable concentration. In one embodiment, the cells are present at between about 20 and about 10⁷ cells/ml, between about 20³10⁶ cells/ml, between about 10⁴ and about 10⁷ cells/ml, between about 10⁴ and about 10⁶ cells/ml, about 20³ and about 10⁵ cells/ml, or between about 10⁵ and about 10⁷ cells/ml. In specific embodiments, the cells are present at between about 10⁴ and about 10⁷ cells/ml, or between about 10⁴ and about 10⁶ cells/ml.

In various embodiments, the cross-linker comprises a cross-linker selected from the group consisting of calcium chloride and thrombin, or a combination thereof, and/or the stabilizer is tranexamic acid. In a specific embodiment, the cross-linker comprises calcium chloride present at a concentration of between about 0.5 mg/ml and about 10 mg/ml, between about 0.5 mg/ml and about 7.5 mg/ml, between about 0.5 mg/ml and about 5 mg/ml, between about 1 mg/ml and about 10 mg/ml, between about 1 mg/ml and about 7.5 mg/ml, between about 1 mg/ml and about 5 mg/ml, between about 1.25 mg/ml and about 10 mg/ml, between about 1.25 mg/ml and about 7.5 mg/ml, or between about 1.25 mg/ml and about 5 mg/ml in the mixture (or the resulting cross-linked scaffold). In another specific embodiment, the cross-linker comprises thrombin at a concentration of between about 0.1 mg/ml and about 5 mg/ml, between about 0.25 mg/ml and about 5 mg/ml, or between about 0.5 mg/ml and about 5 mg/ml in the mixture (or the resulting cross-linked scaffold). In one specific embodiment, the cross linker comprises calcium chloride; in another specific embodiment, the calcium chloride is present at a concentration of between about 1.25 mg/ml and about 5 mg/ml in the mixture or resulting cross-linked scaffold.

In another embodiment, the stabilizer comprises tranexamic acid present at a concentration of between about 1 mg/ml and about 5 mg/ml , between about 2 mg/ml and about 5 mg/ml, or between about 2.5 mg/ml and about 5 mg/ml, in the mixture (or the resulting cross-linked scaffold).

The plasma, crosslinker, and stabilizer may be mixed in a separate container and then aliquoted into multiple wells for cross-linking as deemed appropriate for an intended use. In various embodiments, the plasma, crosslinker and stabilizer may be aliquoted into microtiter wells (for example, 24-well, 48-well, or 96-well plates), well chambers, or capsules prior to cross-linking

Any suitable incubating conditions may be used that lead to cross-linking. In one embodiment, the cross-linking incubation is carried out at about room temperature. The incubating can be carried out for any suitable period of time to accomplish the desired amount of cross-linking. In various embodiment, the cross-linking incubating is carried out for between about 5 minutes to about 8 hours, about 5 minutes to about 6 hours, about 5 minutes to about 4 hours, about 5 minutes to about 2 hours, about 30 minutes to about 8 hours, about 30 minutes to about 6 hours, about 30 minutes to about 4 hours, about 30 minutes to about 2 hours; about 1 hour to about 8 hours, about 1 hour to about 6 hours, about 1 hour to about 4 hours, about 1 hour to about 2 hours, about 2 hours to about 8 hours, about 2 hours to about 6 hours or about 2 hours to about 4 hours.

In another embodiment, no exogenous polymer is present in the three-dimensional cross-linked scaffold, which minimizes the manipulation of the natural development microenvironment provided by the scaffolds of the disclosure. In another embodiment, one or more other polymers may be added as appropriate for an intended use, including but not limited to increasing stiffness of the scaffold. In this embodiment, three-dimensional cross-linked scaffolds can recapitulate soft or stiff tissue characteristics.

The cord blood plasma may be present in the mixture at any suitable concentration. In various embodiments, the cord blood plasma is present in the mixture at a concentration of between about 30% v/v and about 80% v/v, about 30% v/v and about 70% v/v, about 30% v/v and about 60% v/v, or between about 30% v/v and about 50% v/v.

After cross-linking, cell culture media may be added to the scaffold and the scaffolds further incubated for cell growth and any uses, including but not limited to those disclosed herein. Any cell culture medium suitable for the biological cells in the scaffold may be used. The medium may be added to the top of the scaffold, may be added through the wall of the well (i.e.: not directly on top of the 3D culture), or may be added to the scaffold in any other suitable manner.

In one non-limiting embodiment, the plasma from umbilical cord and the resulting scaffolds with biological cells may comprise adding a second population of cells to the top of the scaffold and culturing the second population of cells on the scaffold. In one non-limiting embodiment, the second population may comprise stromal cells (i.e.: mesenchymal, endothelial, immune cells including but not limited to T cells, B cells, NK cells, myeloid-derived suppressor cells and monocytes). In this embodiment, the effect on the second population of cells on cells within the scaffold (cell-cell interactions or cell-ECM production) can be tested in the presence or absence of test compounds. In these embodiments, the second population of cells can be used to recreate different tissue-specific cellular niches.

In another embodiment, post-cross-linking steps, such as adding cell culture medium, cell proliferation/differentiation, and the recited uses, may be carried out at between about room temperature and about 37° C.

In a second aspect, the disclosure provides three-dimensional cross-linked scaffolds made by the method of any embodiment or combination of embodiments of the first aspect of the disclosure.

In a third aspect, the disclosure provides three-dimensional cross-linked scaffolds comprising cord blood plasma. The cord blood plasma may be obtained from any suitable source, including but not limited to a subject that has maternal pregnancy complications such as Type 1, Type 2 or gestational diabetes, preeclampsia, maternal obesity, smoking, multiple gestation, or preterm labor. The subject may also have fetal pregnancy complications such as birth defects, chromosomal or hereditary disorders or intrauterine growth disturbance.

In one embodiment, the scaffold further comprises biological cells within the scaffold. Any suitable biological cells may be used as deemed appropriate for an intended use. In one embodiment, normal or aberrant stem cells from any suitable source can be used, including but not limited to inducible pluripotent stem cells (iPSC), embryonic, fetal, hematopoietic, mesenchymal, bone marrow derived, umbilical cord derived, or placenta derived stem cells in order to test mechanisms of normal or abnormal biologic development or screen therapeutic compounds for efficacy or developmental toxicity. In one embodiment, the biological cells comprise mesenchymal stem cells, including but not limited to human mesenchymal stem cells, including, but not limited to those obtained from umbilical cord including that from the same or other subject.

In some embodiments, the cells and cord plasma are matched (i.e.: from the same subject). They may also be unmatched plasma and biological cells, or matched or unmatched combinations of plasma and biological cells from more than one subject may be used. In this embodiment, the resulting three-dimensional cross-linked scaffolds can be used, for example, for drug screening, tissue engineering, cell differentiation, toxicology studies including reproductive toxicology/teratogenicity studies, cell fate studies based on exposure to stimuli, inherent cell abnormalities, developmental biology, developmental origins of disease, regenerative medicine, etc.

In one embodiment, the biological cells are present in the scaffold at a concentration between about 20³ cells/ml and about 10⁷ cells/ml, between about 20³-10⁶ cells/ml, between about 10⁴ and about 10⁷ cells/ml, between about 10⁴ and about 10⁶ cells/ml, about 20³ and about 10⁵ cells/ml, or between about 10⁵ and about 10⁷ cells/ml. In specific embodiments, the cells are present at between about 10⁴ and about 10⁶ cells/ml.

In one embodiment, the three-dimensional cross-linked scaffold comprises a cross-linker selected from the group consisting of calcium chloride, thrombin, or a combination thereof. In various embodiments, the three-dimensional cross-linked scaffold comprises (i) calcium chloride present at a concentration of between about 0.5 mg/ml and about 10 mg/ml, between about 0.5 mg/ml and about 7.5 mg/ml, between about 0.5 mg/ml and about 5 mg/ml, between about 1 mg/ml and about 10 mg/ml, between about 1 mg/ml and about 7.5 mg/ml, between about 1 mg/ml and about 5 mg/ml, between about 1.25 mg/ml and about 10 mg/ml, between about 1.25 mg/ml and about 7.5 mg/ml, or between about 1.25 nm/ml and about 5 mg/ml; (ii) thrombin at a concentration of between about 0.1 mg/ml and about 5 mg/ml, between about 0.25 mg/ml and about 5 mg/ml, or between about 0.5 mg/ml and about 5 mg/ml in the mixture (or the resulting cross-linked scaffold), or (iii)) combinations thereof. In one specific embodiment, the cross linker comprises calcium chloride; in another specific embodiment, the calcium chloride is present at a concentration of between about 1.25 mg/ml and about 5 mg/ml in the mixture or resulting cross-linked scaffold.

In another embodiment, the scaffold comprises a stabilizer. In one embodiment, the stabilizer comprises tranexamic acid present at a concentration of between about 1 mg/ml and about 5 mg/ml , between about 2 mg/ml and about 5 mg/ml, or between about 2.5 mg/ml and about 5 mg/ml.

In a further embodiment, no exogenous polymer is present in the three-dimensional cross-linked scaffold. In another embodiment, the cord blood plasma is present in the mixture at a concentration of between about 30% v/v and about 80% v/v, about 30% v/v and about 70% v/v, about 30% v/v and about 60% v/v, or between about 30% v/v and about 50% v/v.

In all embodiments disclosed herein, the three-dimensional cross-linked scaffold may be of any suitable thickness. In various embodiments, the three-dimensional cross-linked scaffold has a thickness of between about 100 μm and about 1000 μm, between about 100 μm and about 900 μm, between about 100 μm and about 800 μm, between about 100 μm and about 700 μm, between about 100 μm and about 600 μm, between about 100 μm and about 500 μm, between about 100 μm and about 400 μm, between about 200 μm and about 1000 μm, between about 200 μm and about 900 μm, between about 200 μm and about 800 μm, between about 200 μm and about 700 μm, between about 200 μm and about 600 μm, between about 200 μm and about 500 μm or between about 200 μm and about 400 μm.

In another embodiment, a stiffness of the scaffold ranges between about 0.25 kPa to 2 kPa, between about 0.5 kPa to about 2 kPa, between about 0.75 kPa to about 2 kPa, between about 1 kPa to about 2 kPa, between about 1.25 kPa to about 2 kPa, or between about 1.5 kPa to about 2 kPa. Stiffness can be chemically-induced, or may be modified via the cells.

In another embodiment, the three-dimensional cross-linked scaffolds comprise a porous structure with a network of interconnecting fibrinogen fibers. This embodiment aids, for example, in gas diffusion, nutrient supply, and waste removal through the 3D scaffold. In embodiments in which the scaffolds contain other biological cells, the fibers may further comprise extracellular matrix fibers secreted by the cells, including but not limited to collagen. The main regulator of porosity is the fibrinogen content, but porosity can also be modulated with the crosslinkers and other chemical-inducers or by incorporating other proteins (extracellular matrix, such as collagen, laminin, etc). In various embodiments, the porosity is between about 20 μm and about 100 μm, between about 20 μm and about 75 μm, or between about 20 μm and about 50 μm in diameter. In a specific embodiment, the porosity is between 2 μm and about 8 μm in diameter.

In a fourth aspect, the disclosure provides uses of the three-dimensional cross-linked scaffold of any embodiment of combination of embodiments disclosed herein for any suitable purpose, including but not limited drug screening, tissue engineering, cell differentiation, toxicology studies including reproductive toxicology/teratogenicity studies, cell fate studies based on exposure to stimuli, inherent cell abnormalities, developmental biology, developmental origins of disease, regenerative medicine, etc.. In one embodiment, such use may comprise

(a) contacting the three-dimensional cross-linked scaffold with a test moiety, wherein the test moiety may include, but is not limited to a drug, toxin, hormone, cytokine, small molecule, and/or other stimulus;

(b) culturing the cells of interest within and/or on top the scaffold; and

(c) determining an effect of the test moiety on the cells of interest.

As discussed above, after cross-linking, cell culture media may be added to the scaffold and the scaffolds further incubated for cell growth and any uses, including but not limited to those disclosed herein. Any cell culture medium suitable for the biological cells in the scaffold may be used. The medium may be added to the top of the scaffold, may be added through the wall of the well (i.e.: not directly on top of the 3D culture), or may be added to the scaffold in any other suitable manner.

In one embodiment, cord plasma and resulting scaffolds from normal (non-complicated pregnancy) and abnormal pregnancy may be used to study cellular responses following exposure to normal or abnormal circulating factors including, but not limited to nutrients, fuels, hormones, cytokines, adipokines, eicosanoids, or hormones. In another embodiment, normal or abnormal cord blood plasma with circulating or added drug compounds or small molecules can be used to test responses of normal or abnormal stem cells to potential therapeutics or toxicants under variable developmental conditions.

### EXAMPLES

### iWOMB: Human Model for Precision Based Developmental and Reproductive Assays

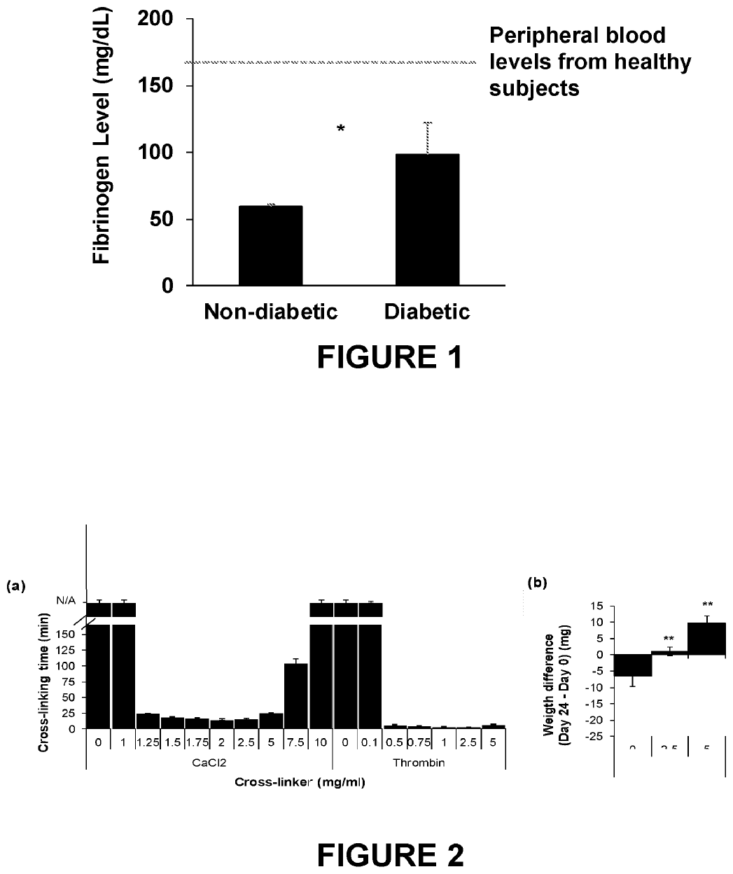

Referring to FIG. 1, cord blood was analyzed for fibrinogen content through the clotting method of Clauss. The Clauss fibrinogen assay is a quantitative, clot-based, functional assay. The assay measures the ability of fibrinogen to form fibrin clot after being exposed to a high concentration of purified thrombin. Fibrinogen content characterization in cord blood showed a low fibrinogen content level in cord blood revealing a unique milieu when compared to other plasma sources such as periphearl blood. In addition, cord blood plasma from diabetic moms showed higher levels than no-diabetic moms highlighting precision-based applications.

Referring to FIG. 2, cross-linking time was assessed by measuring the time necessary to achieve matrix cross-linking using three relevant cross-linkers of the blood coagulation process including thrombin (0-5 mg/ml) and CaCl₂ (0-10 mg/ml). The stabilization effects of preventing fibrin degradation and stability improvement in the scaffold was assessed by surveying an antifibrinolytic agent such as tranexamic acid (0-5 mg/ml). The stability of the scaffold was studied by measuring each scaffold weight at day 0 and again measuring scaffold weight at the conclusion of a 3 week time period. Chemical characterization of cord blood plasma allowed the optimization for controlled cross-linking capabilities and prevention of degradation. CaCl₂ (1.25 to 5 mg/ml) and thrombin (0.5 to 5 mg/ml) showed the fastest crosslinking. Tranexamic acid in the range of 5 mg/ml revealed the best improvement in scaffold stability.

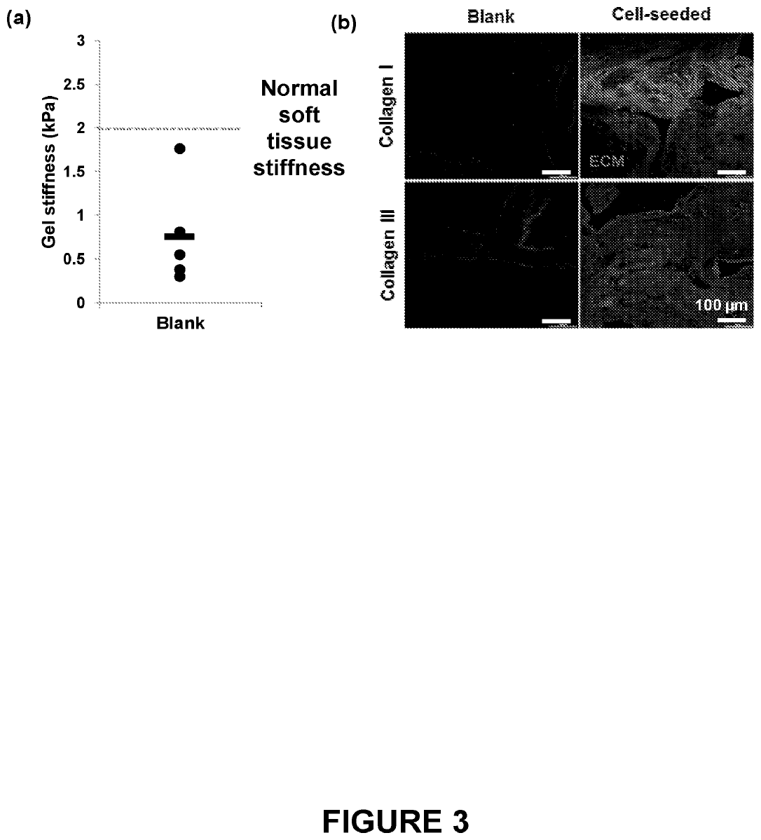

Referring to FIG. 3, the stiffness of the scaffolds was measured by atomic force microscopy (AFM). The Young's modulus was estimated by fitting a modified Hertz model onto the AFM indentation curve using the built in function of AFM software (Asylum Research). These scaffolds were also fixed and processed on a Leica™ 300 ASP tissue processor. Paraffin-embedded 3D matrix sections were longitudinally sliced at 10 μm then stained for anti-collagen-I and anti-collagen-III. A FITC conjugated secondary antibody was used whenever applicable. Stiffness assessment revealed a soft gelatinous-like blank acellular scaffold with values of 0.75 kPa when compared to soft tissue stiffness of about 2 kPa. When cells are incorporated, scaffolds revealed an increased extracellular matrix (ECM) proteins secretion including collagen I and collagen III, relevant ECM proteins of soft tissue, in comparison to blank acellular gels. These results highlight the physical properties of iWOMBs and optimization can be performed by controlling cell seeding.

Human and non-human stein cells were incorporated in or seeded on human cord plasma derived three-dimensional cross-linked scaffolds to establish applications for regenerative medicine, tissue engineering, reproductive toxicology/teratogenicity studies, developmental biology, and developmental origins of disease. Fresh and bio-banked samples were collected under oversight by the Sanford Health Institutional Review Board. Specifically, umbilical (venous) cord blood and cord tissue were collected from consenting maternal donors between the ages of 18-45 years who delivered by cesarean section (n=179 subjects). Umbilical venous blood was collected by gravity into a sterile collection bag containing citrate anti-coagulant after infant delivery and cord clamping. Plasma was separated and stored at −80° C. until used to make cross-linked fibrin matrices for iWOMB. Fresh cord tissue was rinsed in iced saline and transported in sterile saline for processing the same day. Under sterile conditions, vessels were removed and the remaining tissue was minced. Human umbilical mesenchymal stem cells (hu-MSC) were derived from the Wharton's jelly by explant method or overnight digestion in collagenase type IV followed by a secondary digestion in trypsin. Hu-MSC were expanded to 70-85% confluency, aliquoted and cryopreserved in vapor phase until use. By both explant and digestion method, isolated hu-MSC meet international standards for stem cells: adhere to plastic in standard culture conditions and have >95% expression of MSC markers CD90, CD105, and CD73 by flow cytometry with little to no expression of hematopoietic or endothelial cell markers CD45, CD19, CD31, and CD34. Thawed, hu-MSC maintain self-renewal capabilities (see previous supplemental data) and are multipotent (see FIG. 6).

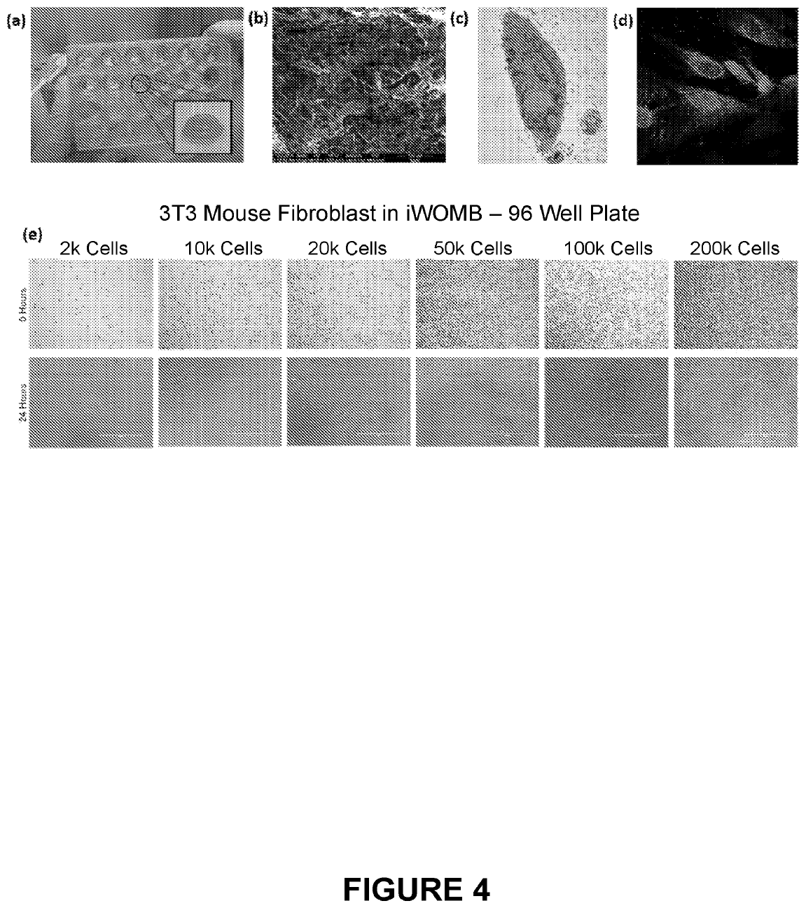

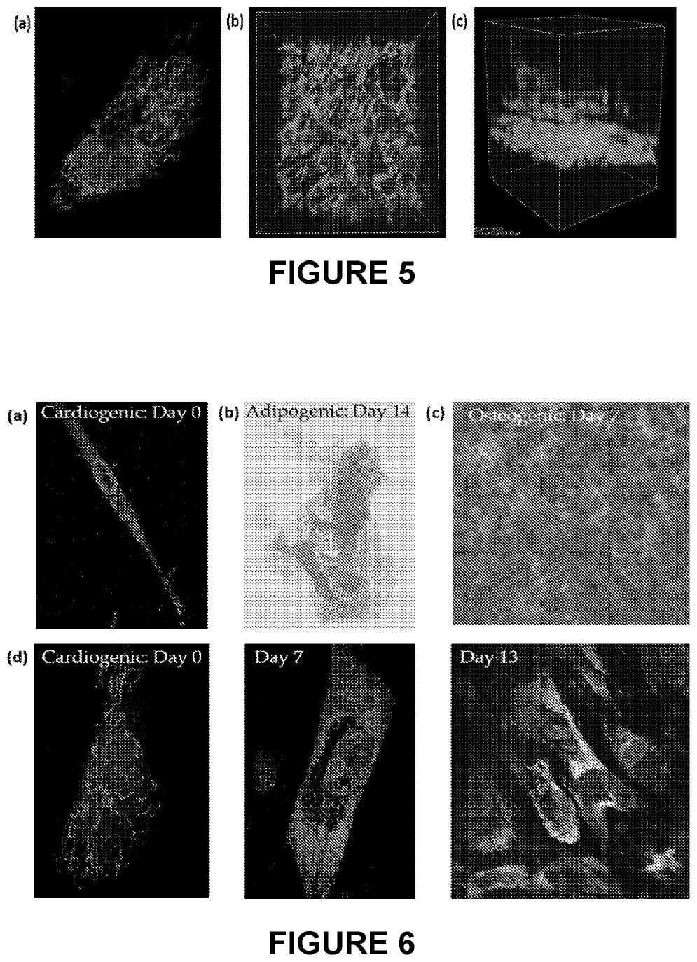

Hu-MSCs were plated on cord plasma derived three-dimensional scaffolds to evaluate cell properties (FIG. 4), cell-cell and cell-matrix interactions (FIG. 5) and cell fate in the mixture (FIG. 6). Hu-MSC from the same (matched) and different (unmatched) subjects were evaluated in the mixture. To establish iWOMB stability in a variety of media, hu-MSC were cultured at 37° C. and 5% CO₂ within three-dimensional cross-linked scaffolds as follows:

1) Stem cell maintenance media: Alpha Modification of Eagle™'s Medium (αMEM; ThermoFisher, MT15012CV), 10% Fetal Bovine Serum (FBS; Hyclone, SH3039603FBS), 1% penicillin/streptomycin (Hyclone™, SV30010), 1% L-Glutamine (Sigma Aldrich, G7513-100 ml) with or without 250 uM Amphotericin B (Sigma Aldrich, A2942-20 ML)

2) StemPro™ Adipogenesis Differentiation media (Gibco, A10070-01)

3) StemPro™ Osteogenesis Differentiation media (Gibco, A10072-01)

4) PSC Cardiomyocyte Differentiation Media: A, B and Maturation media (Gibco, A2921201).

To establish stability and function of iWOMB three-dimensional cross-linked scaffolds for a variety of cells, non-human T3T primary mouse embryonic fibroblast (NIH/3T3 ATCC® CRL1658™ ) cells were seeded on iWOMB at varying seeding densities in stem cell maintenance media. Images were captured just after seeding and after 24 hours in culture for morphological investigation and to detect optimal seeding for cell-cell and cell-matrix interactions without die off from overcrowding.

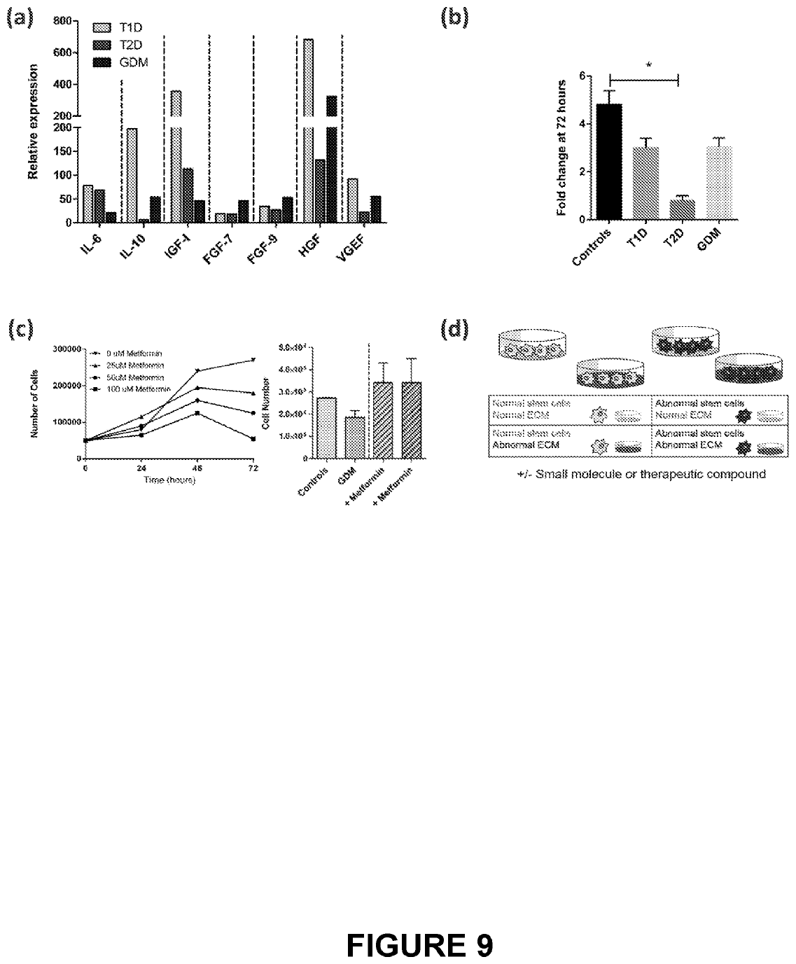

A variety of cells, including human and non-human stein cells are supported by iWOMB three-dimensional cross-linked scaffolds. Both matched (same subject) and unmatched (different subject) hu-MSC grow well in umbilical cord plasma derived scaffolds allowing cross-over studies for precision-based developmental biology and programming applications (FIG. 9). Cells incorporate and remain viable in iWOMB at varying seeding densities (FIG. 4e) and in a variety of media including growth and differentiation media. Despite its gelatinous nature, hu-MSCs incorporate into iWOMB and retain normal sub-cellular structure and organelle function, as shown in FIG. 4c-d, 5 and 6. Cells within scaffolds can be imaged fixed (FIG. 4c) or live (FIGS. 4d, 5 and 6).

Referring to FIG. 5, Hu-MSCs were grown until confluent then treated with 10 uM 5-azacytadine (Sigma, St. Louis, Mo.) for 24 hrs. Cells were allowed to recuperate for 24 hrs in stem cell media (αMEM, 10% FBS, 1% L-glutamine, 1% penicillin/streptomycin) then 20,000 hu-MSC were plated in 1000 μl scaffolds within 24-well plates and incubated in StemPro™ Cardiogenic Differentiation media B for 2 days followed by M (maturation) media for the remaining time (ThermoFisher, Waltham, Mass.). Media changes were every 2-3 days according to the manufacturer's directions. iWOMBs were stained 1.43 uM MitoTracker™ green (M7514, Thermo Fisher Scientific) to identify mitochondria, 20 nM tetramethylrhodamine ethyl ester (TMRE) Red (T669, Thermo Fisher Scientific) to identify mitochondrial membrane potential in for ATP production, 2 uM LysoTracker™ blue or red as noted in figure legends (ThermoFisher, Waltham, Mass.), and 1:200 Hoescht (AS-83218, AnaSpec Inc.) as above. Images were acquired at 60× using a Nikon AIR Confocal microscope and NIS Elements Software. Three-dimensional umbilical cord plasma-derived scaffolds support stem cell growth and differentiation in culture under various conditions that include drug treatment and multiple media changes for cardiogenic differentiation. Three dimensional organization is retained. Cell-to-cell, cell-to-media, and cell-to-matrix interactions are maintained in tissue like organization.

To demonstrate the usefulness of iWOMB for tissue engineering, reproductive toxicology/teratogenicity studies, developmental biology, developmental origins of disease, and regenerative medicine, hu-MSC were differentiated to cardiac, adipogenic and osteogenic lineages in three-dimensional cord plasma derived scaffolds. Cardiogenesis in iWOMB is described in detail above (FIG. 5). Specifically here, hu-MSC were treated with 5-AZA and incorporated into unmatched umbilical cord plasma derived scaffolds (different subjects) and imaged at various stages during differentiation (day 2, 5, 7, 14 and 21 post differentiation). To determine if hu-MSC developed into cardiac lineage, cell seeded iWOMB in a 35 mm glass bottom FluoroDish™ (FD3510, World Precision Instruments) were fixed using 4% paraformaldehyde then incubated in 1:100 myosin light chain 2 (MLC2v) primary antibody (rabbit, AbCam) and cardiac troponin (TNNT2) primary antibody (mouse, AbCam) overnight followed by incubation with 1:250 Rb488 (ThermoFisher) and Ms594 (ThermoFisher) secondary antibodies for 2 hrs. Samples were incubated in 1:200 DAPI solution for 30 min prior to imaging. To further define morphology and sub-cellular characteristics that are consistent with myocytes, iWOMB used for cardiogenesis were also stained with 1.43 uM MitoTracker™ green, 30 nM TMRE (ThermoFisher, Waltham, Mass.) and 1:200 Hoescht (AS-83218, AnaSpec Inc.). Images were acquired using a Nikon AIR Confocal microscope at 60× magnification with NIS elements software. At the same time-points, cell-seeded iWOMBs were placed in OCT and frozen at −20° C. before being sectioned and stained with Oil Red O (Sigma) for 30 min. Images were taken using a Nikon 90i light microscope at 60× magnification. Representative images were taken using a Nikon 90i light microscope at 60× magnification. Hu-MSCs were pushed towards osteogenic differentiation using the StemPro™ Osteogenic Differentiation kit (ThermoFisher, Waltham, Mass.) according to manufacturer's protocol. At the same time-points, cell-seeded iWOMBs were fixed using 4% paraformaldehyde then stained with a 2% Alizarin Red S solution (Sigma) for 20 mins. Images were taken using a Nikon 90i light microscope. To further define morphology and sub-cellular characteristics that are consistent with myocytes, iWOMBs uused for cardiogenesis were live cell imaged in a 4-well glass chamber slide and stained with 1.43 uM MitaTracker' green, 30 nM TMRE (ThermoFisher, Waltham, Mass.), and 1:200 Hoescht (AS-83218, AnaSpec Inc.). Images were taken using a Nikon AIR Confocal microscope at 60× magnification using NIS elements software.

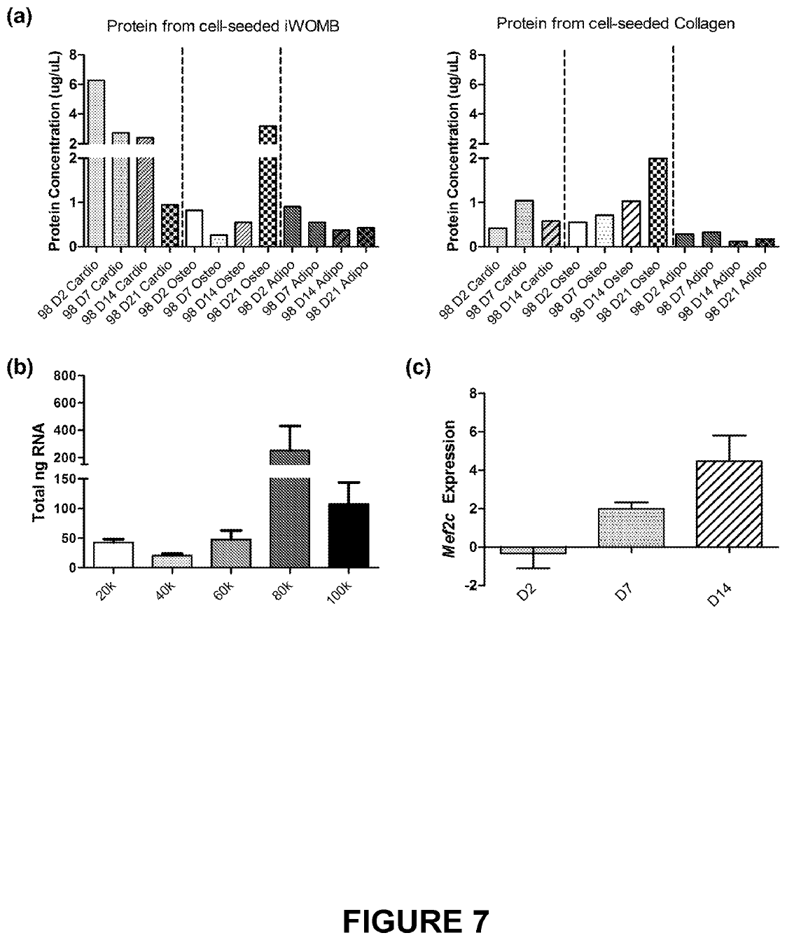

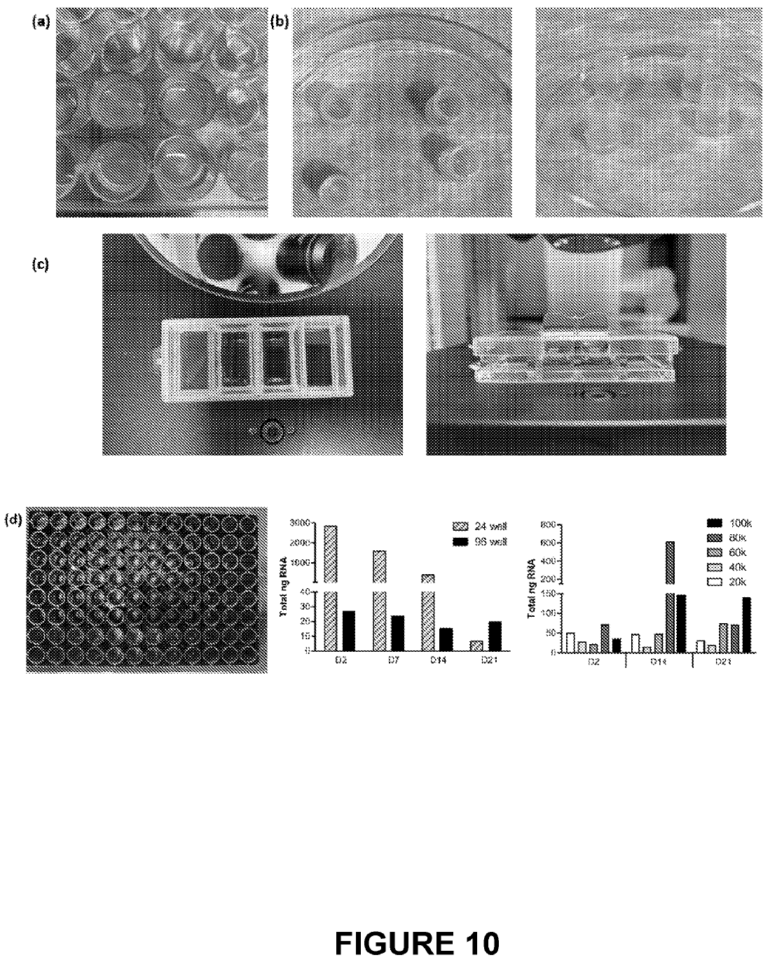

Hu-MSC in iWOMBs proliferate and undergo cardiogenic, adipogenic, osteogenic differentiation by standardized techniques. Cardiogenesis yields bi-nucleated, rod-shaped, cardiomyocyte precursors which stain positive for myosin light chain 2 (MLC2) and cardiac troponin (TNNT2) (FIG. 6a). By 2 weeks post-differentiation by these methods, cardiac progenitors increasingly express cardiomyocyte-specific lineage markers (FIG. 7c) and develop subcellular organization of well-described mitochondrial sub-sets that are specific to myocytes. These include long, poorly charged MitoTracker™ green stained perinuclear mitochondria and highly-charged, ATP producing interfibrillar mitochondria that appear gold mitochondria due to co-localized MitoTracker™ green and TMRE red. Adipogenesis yields cells with a high number of Oil-red-O (red) lipid droplets that accumulate between 7-14 days post-differentiation (FIG. 6b). Osteogenesis yields visible calcium deposition within the wells (FIG. 10a) and densely packed cells within scaffolds that stain intensely positive with alizarin red by 7 days post-differentiation (FIG. 6c).

7) We tested the ability to isolate protein and RNA from cell-seeded iWOMBs for molecular analyses. To limit patient to patient variables, hu-MSC from one subject (98) were cultured on unmatched cord blood derived scaffolds or collagen coated 24 well plates and then differentiated in to multiple lineages as described above. On advancing days 2, 7, 14 and 21 post-differentiation (D), cells were collected by collagenase I digestion for iWOMB or trypsinization for collagen. Cells in culture were pelleted and protein was isolated by trituration in RIPA lysis and extraction buffer. Cell lysate protein was quantified by DC Protein Assay (BioRad, Hercules, Calif.). RNA electropherograms were assessed and concentrations were measured by Epoch spectrophotometer (BioTek, Winooski, Vt.). To validate RNA isolation for variable assays, hu-MSC were mixed in three-dimensional cord plasma derived scaffolds in 96-well plates at increasing seeding density of 20K, 40K, 60K, 80K and 100K cells/scaffold. Cells were pellets as previously described and RNA was isolated using RNeasym™ Micro kit (Qiagen, Geimantown, Md.). RNA integrity was assessed by electropherograms using 2100 BioAnalyzer (Agilent Technologies, Santa Clara, Calif.) and RNA concentration was measured by Epoch spectrophotometer (BioTek, Winooski, Vt.). Using 1 ug of RNA, complementary DNA (cDNA) was synthesized using iScript™ cDNA Synthesis Kit and T100 Thermal Cycler (Bio-Rad, Hercules, Calif.) via manufacturer's protocol. Quantitative PCR (qPCR) was performed by TaqMan™ approach in an ABI7500 qPCR system with Absolute Blue™ qPCR Mix (ThermoFisher, Waltham, Mass.). Beta-2-microglobulin (B2M) or Ribosomal Protein Lateral Stalk Subunit PO (RPLP0), which remain stable over the course of differentiation were used as the reference genes.

Protein and RNA can be successfully isolated from cell-seeded iWOMBs. Protein collected from cell-seeded three-dimensional cross-linked scaffolds was typically greater or equal to protein recovered from collagen coated plates. Protein and RNA concentrations reflect cell numbers including during cardiogenic, osteogenic and adipogenic differentiation (FIG. 7). For example, terminally differentiated cardiac progenitors do not proliferate and decline with cardiogenesis, so do protein concentration. Conversely, hu-MSC number initially declines with osteogenesis induction but then dividing osteocyte progenitors proliferate over time, thus protein content increases between D7 and 21. RNA increases with seeding density until overcrowding occurs. Protein can be used to study the developmental microenvironment (DME) or cellular protein expression. RNA can purified and used for PCR to detect expression of lineage-specific developmental markers over time or confirm genetic or genomic variation between cells.

To determine the combined contribution of cells, media and the extracellular matrix (ECM) to the developmental microenvironment (DME) within iWOMBs, we measured cytokines and growth factors collected from acellular and cell-seeded three-dimensional cord plasma-derived scaffolds and compared relative expression differences over the course of hu-MSC cardiogenesis. Hu-MSC plated to three-dimensional cord plasma derived scaffolds underwent cardiogenic differentiation and protein was collected from cells and supernatant at baseline and on differentiation day 2, 5, 7, and 10 as detailed above (FIGS. 5 & 7). Using a custom human antibody array (Ray Biotech, Peachtree Corners, Ga.), we measured supernatant proteins tumor necrosis factor alpha (TNFα), interleukins (IL-6, IL-10), insulin-like growth factor (IGF-1), fibroblast growth factor (FGF-7, FGF-9), hepatocyte growth factor (HGF), and vascular endothelial growth factor (VEGF) which were run in duplicate. Specifically, 100 ug of protein was incubated on the custom dot-blot membranes overnight. Following the manufacturer's instructions, the membranes were exposed for 2 minutes on a LiCOR Odyssey™ imager. Densitometry analysis was performed using UVP VisonWorks™ LS software and recorded as expression relative to membrane controls (to account for membrane background). Comparison of protein in cell lysate and in the supernatant within each well was done by T-test. The difference in protein expression over time was done by analyzing differences in relative protein expression among baseline day 0 (acellular iWOMB+media) and each day 2, 5, 7, and 10 by one-way ANOVA and Dunnett post-test analysis.

Proteins within the DME can be measured in both cell lysate and supernatant from acellular and cell-seeded iWOMB. Protein within the DME of iWOMB is dynamic over the course of differentiation. Variables affecting the DME include the cord plasma derived ECM itself (FIG. 9), cytokines and growth factors in various media, and cytokines and growth factors secreted by the cells in the organized culture. Specifically, the addition of hu-MSC to the scaffold leads to an immediate and sustained increase in IL6 and introduces a cell source for TNFα, IL10, and HGF which increase steadily with the number of days cells are in culture. An additional difference in DME during cardiogenesis comes from the media. Specifically, there is a high amount of FGF-7 and 9 in baseline media on day 0. Changing to cardiogenic media B (first measured on day 2) incites cytokine production (TNF, IL6, IL10); this is not surprising as cells appear stressed after this change. Cardiogenic media also has different growth factors that are important for each step of cardiogenesis. This is noticeable as the DME has less FGF and more IGF I and VEGF after these transitions.

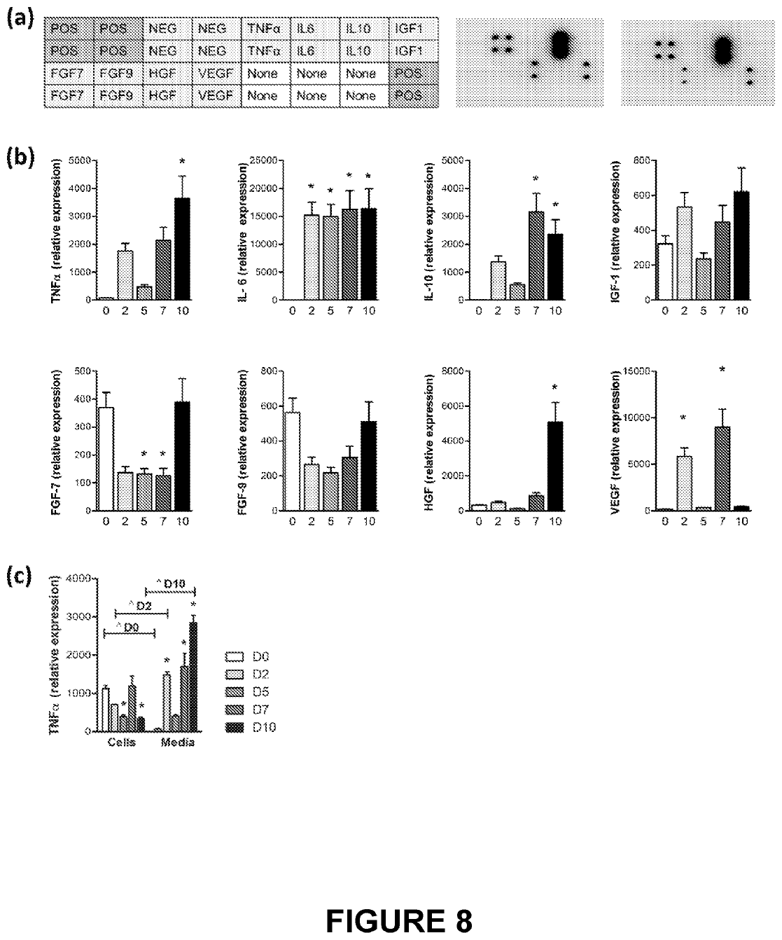

To determine whether iWOMB is a useful tool for precision-based assays, we used umbilical cord plasma and hu-MSC from control and diabetic pregnancy to identify diabetes-related differences in proteins in the DME and/or programmed cell fate. Because diabetic pregnancy varies significantly based on underlying mechanisms, we used samples from subjects with Type 1 (T1D), Type 2 (T2D) and gestational diabetes (GDM). Using our customized antibody array as detailed above (FIG. 8), we compared relative expression of cytokines and growth factors in cord plasma protein. Just as fibrinogen levels are higher in cord plasma from diabetic pregnancies, so are other factors. Specifically, umbilical cord plasma cytokines (IL-6, IL-10), insulin like growth factor −1 (IGF-1) and hepatocyte growth factor (HGF) from Type 1 diabetic pregnancy (TID) are higher than in plasma from Type 2 (T2D) and gestational diabetes (GDM) (FIG. 9a). To detect inherent cellular differences (not related to factors in plasma), 50K hu-MSCs/well (n=2 subjects in paired replicates) were plated in stem cell media on collagen coated 24-well plates. Every 24 hours cells were detached using 0.25% trypsin/EDTA and live cells were counted using Trypan blue staining and a hemocytometer. Live cells were recorded at each time point for 96 hours. Media was not changed for the duration of the growth curve experiment. Findings suggest that hu-MSC from diabetic mothers have slower growth, especially those exposed to T2D (FIG. 9b). To test in vivo cell responses to potential therapeutic agents, we repeated the growth assay using control and diabetes-exposed hu-MSC plated in stem cell media supplemented with increasing concentrations of metformin (0 uM, 25 uM, 50 uM, 100 uM). Every 24 hours cells were detached using 0.25% trypsin/EDTA and live cells were counted using Trypan blue and a hemocytometer. Live cells were recorded at each time point for 72 hours. Media was not changed for the duration of the experiment. Normal hu-MSC treated with metformin have a dose-dependent decline in cell growth (FIG. 9c). A similar growth experiment was done using hu-MSC from control and GDM subjects (20K hu-MSCs/well; n=2-3/group). Media was supplemented with 25 μM metformin which is an approximate level reported in cord blood from women taking oral metformin during pregnancy. Exposure to metformin levels reported in umbilical cord blood, does not impair growth of control hu-MSC, and growth of GDM exposed hu-MSC actually improves which suggests programmed stein cells respond differently to the drug.

The data demonstrate that iWOMB is a useful tool for precision-based assays. Using various combinations of normal and abnormal cord plasma derived ECM scaffolds (DME) and stem cells as shown in FIG. 9d offers high-throughput, translational, human assays to understand mechanisms of developmental programming, regenerative medicine, developmental biology, and precision-based pharmacotherapeutics and developmental and reproductive toxicology (DART) screening.

Validation experiments were done to test various applications of the iWOMB. Differentiation is performed with ease in 24-well plates (FIG. 10a). Umbilical cord plasma was combined with hu-MSCs, stem cell media, crosslinking, and stabilizer solutions. In a 24-well plate, 1 ml of the combined solution containing 200,000 cells was aliquoted into each well and was allowed to crosslink for approximately 10 min. Crosslinking was confirmed by holding plate at a 90° angle proceeded by holding the plate upside down for approximately 10 sec. Once confirmed, lint of stem cell media was gently added to the tops of the cross-linked cell-seeded scaffold for 24 hours. The following day, stem cell media was replaced with StemPro™ Osteogenesis Differentiation media (Gibco, A10072-01) to induce osteogenic differentiation. The differentiation media was changed every 3-4 days. Beem® capsules are ideal for fixed imaging or tissue regeneration studies where specified orientation is necessary (FIG. 10b). Using a scalpel, the closed end of the Beem® capsules were removed before the tops were capped and parafilmed to prevent leaking. The Beem® capsules were sterilized under UV light for 1 hour before being placed in a 24-well plate cap side down. 300 ul of iWOMB solution was aliquoted into each Beem® capsule and allowed to crosslink for approx. 10 min. The Beem® capsule was inverted for approximately 5 sec to confirm crosslinking. After confirmation, 300 ul of media was added to the top of the iWOMB. Media was changed every 2-4 days. Pink color in the image indicates fresh media was applied (lower right). (c) Chamber slides are useful for confocal live cell imaging or videos. Using a Lab-Tek 4-well glass chamber slide, 1 ml of pre-cross-linked iWOMB solution was aliquoted into each well. The solution was allowed to crosslink for an additional 10 min before holding the slide at a 90° angle. After confirmation of crosslinking, 1 ml of stem cell media was gently added to the tops of the iWOMB. For imaging, iWOMBs can be fixed in 4% paraformaldehyde for 20 mins before storage at 4° C. (d) Less plasma is needed for 96 well plates which allows upscaling, but protein and RNA yield must be considered for each application as noted during this hu-MSC cardiogenic differentiation assay. Here, hu-MSC were seeded at increasing density in 96-well plates. Each well contained 100 μl of the combined solution seeded with a range of cells from 20k/well to 100k/well. The iWOMB solution was allowed to crosslink for approx. 15 minutes to take into account the surface tension of the smaller well size. Gentle prodding with a 200 μl micropipette tip to the top of the iWOMB was done to confirm crosslinking before 150 ul of stem cell media was added. RNA was isolated and concentration was measures as detailed above (FIG. 7). RNA yield varies based on the starting seeding density, well size, and day in culture.

iWOMB mixtures may be aliquoted into a wide variety of microtiter wells (for example, 24-well, 48-well, or 96-well plates), chamber slides or Beem® capsules for a wide variety of applications. Depending on the well size and volume used, the three-dimensional cross-linked scaffold has a thickness of between about 100 μm and about 1000 μm, all which support tested cells. After cross-linking, culture media suitable for the cells and application in the scaffold may be added to the mixture to support cell growth, differentiation or test exposures.

1. A method, comprising:
(a) mixing cord blood plasma, with cross-linker and stabilizer to form a mixture; and
(b) incubating the mixture for a time and under conditions to form a three-dimensional cross-linked scaffold.

2.-18. (canceled)

19. A three-dimensional cross-linked scaffold comprising cord blood plasma.

20. The three-dimensional cross-linked scaffold of claim 19, wherein the scaffold further comprises biological cells within the scaffold.

21. The three-dimensional cross-linked scaffold of claim 19, wherein the cord blood plasma comprises cord blood plasma obtained from a subject having maternal pregnancy complications such as Type 1, Type 2 or gestational diabetes, preeclampsia, maternal obesity, smoking, multiple gestation, or preterm labor, and/or a subject having fetal pregnancy complications such as birth defects, chromosomal or hereditary disorders or intrauterine growth disturbance.

22. The three-dimensional cross-linked scaffold of claim 20, wherein (a) the biological cells comprise normal or aberrant stem cells from any suitable source, including but not limited to inducible pluripotent stem cells (iPSC), embryonic stem cells, fetal stem cells, hematopoietic stem cells, mesenchymal stem cells, bone marrow derived stem cells, umbilical cord derived stem cells, or placenta derived stem cells.

23. The three-dimensional cross-linked scaffold of claim 20, wherein the biological cells are present in the scaffold at a concentration between about 10³ cells/ml and about 10⁷ cells/ml, between about 10³ and about 10⁶ cells/ml, between about 10⁴ and about 10⁷ cells/ml, between about 10⁴ and about 10⁶ cells/ml, between about 10³ and about 10⁵ cells/ml, or between about 10⁵ and about 10⁷ cells/ml.

24. The three-dimensional cross-linked scaffold of claim 19 comprising a cross-linker selected from the group consisting of calcium chloride, thrombin, or a combination thereof.

25. The three-dimensional cross-linked scaffold of claim 24, comprising (i) calcium chloride present at a concentration of between about 0.5 mg/ml and about 10 mg/ml, between about 0.5 mg/ml and about 7.5 mg/ml, between about 0.5 mg/ml and about 5 mg/ml, between about 1 mg/ml and about 10 mg/ml, between about 1 mg/ml and about 7.5 mg/ml, between about 1 mg/ml and about 5 mg/ml, between about 1.25 mg/ml and about 10 mg/ml, between about 1.25 mg/ml and about 7.5 mg/ml, or between about 1.25 mg/ml and about 5 mg/ml, or mixtures thereof.

26. The three-dimensional cross-linked scaffold of claim 19, further comprising tranexamic acid.

27. The three-dimensional cross-linked scaffold of claim 26, wherein the tranexamic acid present at a concentration of between about 1 mg/ml and about 5 mg/ml , between about 2 mg/ml and about 5 mg/ml, or between about 2.5 mg/ml and about 5 mg/ml.

28. The three-dimensional cross-linked scaffold of claim 19, comprising (A) calcium chloride present at a concentration of between about 0.5 mg/ml and about 10 mg/ml, between about 0.5 mg/ml and about 7.5 mg/ml, between about 0.5 mg/ml and about 5 mg/ml, between about 1 mg/ml and about 10 mg/ml, between about 1 mg/ml and about 7.5 mg/ml, between about 1 mg/ml and about 5 mg/ml, between about 1.25 mg/ml and about 10 mg/ml, between about 1.25 mg/ml and about 7.5 mg/ml, or between about 1.25 mg/ml and about 5 mg/ml; and (B) tranexamic acid present at a concentration of between about 1 mg/ml and about 5 mg/ml , between about 2 mg/ml and about 5 mg/ml, or between about 2.5 mg/ml and about 5 mg/ml.

29. The three-dimensional cross-linked scaffold claim 19, comprising (A) comprising calcium chloride present at a concentration of between about 1.25 mg/ml and about 5 mg/ml; and (B) tranexamic acid present at a concentration of between about 2.5 mg/ml and about 5 mg/ml.

30. The three-dimensional cross-linked scaffold of claim 20, comprising cells are present at between about 10⁴ and about 10⁷ cells/ml or between about 10⁴ and about 10⁶ cells/ml.

31. The three-dimensional cross-linked scaffold of claim 20, wherein no exogenous polymer is present in the three-dimensional cross-linked scaffold.

32. The three-dimensional cross-linked scaffold of claim 20, wherein the cord blood plasma is present in the mixture at a concentration of between about 30% v/v and about 80% v/v, about 30% v/v and about 70% v/v, about 30% v/v and about 60% v/v, or between about 30% v/v and about 50% v/v.

33. The three-dimensional cross-linked scaffold of claim 19, wherein the scaffold has a thickness of between about 100 μm and about 1000 μm, between about 100 μm and about 900 μm, between about 100 μm and about 800 μm, between about 100 μm and about 700 μm, between about 100 μm and about 600 μm, between about 100 μm and about 500 μm, between about 100 μm and about 400 μm, between about 200 μm and about 1000 μm, between about 200 μm and about 900 μm, between about 200 μm and about 800 μm, between about 200 μm and about 700 μm, between about 200 μm and about 600 μm, between about 200 μm and about 500 μm or between about 200 μm and about 400 μm.

34. (canceled)

35. The three-dimensional cross-linked scaffold of claim 19, wherein the scaffold has a stiffness of between about 0.25 kPa to 2 kPa, between about 0.5 kPa to about 2 kPa, between about 0.75 kPa to about 2 kPa, between about 1 kPa to about 2 kPa, between about 1.25 kPa to about 2 kPa, or between about 1.5 kPa to about 2 kPa.

36. The three-dimensional cross-linked scaffold of claim 19, wherein the scaffold has a porosity is between about 20 μm and about 100 μm, between about 20 μm and about 75 μm, or between about 20 μm and about 50 μm in diameter.

37. Use of the three-dimensional cross-linked scaffold of claim 19 for any suitable purpose, including but not limited to drug screening, tissue engineering, cell differentiation, toxicology studies including reproductive toxicology/teratogenicity studies, cell fate studies based on exposure to stimuli, inherent cell abnormalities, developmental biology, developmental origins of disease, regenerative medicine, etc.

38. (canceled)
